(19) United States
(12) Patent Application Publication
(10) Pub. No.: US 2026/0224268 A1
(43) Pub. Date: Aug. 6, 2026

(54) SYSTEM AND METHOD FOR TISSUE TREATMENT

(71) Applicant: William Wallander, Dallas, TX (US)

(72) Inventor: William Wallander, Dallas, TX (US)

(21) Appl. No.: 19/046,711

(22) Filed: Feb. 6, 2025

(51) Int. Cl. A61B 18/06; A61B 18/00; A61B 18/18

(52) U.S. Cl. CPC A61B 18/06; A61B 18/18; A61B 2018/0047; A61B 2018/00577; A61B 2018/1807

(57) ABSTRACT

A system for tissue treatments includes a delivery device having an application end and a delivery end. A channel extends between a delivery opening in the delivery end and an application opening in the application end. The application opening is sized to surround a treatment area so that the treatment area is accessible through the channel. The delivery opening is configured to receive a treatment so that the treatment is contained in the channel.

### BACKGROUND

The disclosed embodiments relate to tissue treatments, and more particularly, to tissue treatments having a delivery device.

Generally, skin blemishes are treated using a suitable chemical, such as, liquid nitrogen or nitrous oxide. The chemical is typically applied to a swab that is placed in contact with the blemish. In other examples, the chemical is placed in a container that is positioned adjacent to the skin blemish. In either case, the chemical is either saturated into the swab or merely sits on the skin blemish. Currently treatment devices do not include a means to apply the chemical with significant pressure. Additionally, current treatment devices are not specifically shaped to the skin blemish. Accordingly, in some cases the chemical is not applied entirely to the skin blemish, or the chemical is applied to healthy skin.

### SUMMARY

The present disclosure includes one or more of the features recited in the appended claims and/or the following features which, alone or in any combination, may comprise patentable subject matter.

According to a first aspect of the disclosed embodiments, a system for tissue treatments includes a delivery device having an application end and a delivery end. A channel extends between a delivery opening in the delivery end and an application opening in the application end. The application opening is sized to surround a treatment area so that the treatment area is accessible through the channel. The delivery opening is configured to receive a treatment so that the treatment is contained in the channel. An applicator is configured to be inserted through the delivery opening and moved toward the application opening to apply the treatment contained in the channel against the treatment area.

In some embodiments of the first aspect, the applicator may press the treatment against the treatment area when the applicator is moved toward the application opening. An applicator pad may be located in the application end adjacent the application opening. The applicator pad may absorb the treatment delivered though the channel. The applicator pad may be pressed against the treatment area when the applicator is moved toward the application opening. The application opening may be custom fit to a size and shape of the treatment area. The size and shape of the treatment area may be measured with a mobile device. An ablation tool may be configured to remove at least a portion of the treatment area. The ablation tool may be positioned in the delivery device.

According to a second aspect of the disclosed embodiments, a kit for tissue treatments includes a plurality of delivery devices. Each delivery device includes an application end and a delivery end. A channel extends between a delivery opening in the delivery end and an application opening in the application end. The application opening of each delivery device is sized to surround a treatment area so that the treatment area is accessible through the channel. A treatment container contains a treatment. The treatment container is configured to discharge the treatment through the delivery opening so that the treatment is contained in the channel. An applicator is configured to be inserted through the delivery opening and moved toward the application opening to compress the treatment contained in the channel against the treatment area.

In some embodiments of the second aspect, the treatment may be at least one of liquid nitrogen or nitrous oxide. Each delivery device of the plurality of delivery devices may have a different sized application opening to accommodate different sized treatment areas. The applicator may press the treatment against the treatment area when the applicator is moved toward the application opening. An applicator pad may be located in the application end adjacent the application opening. The applicator pad may absorb the treatment delivered though the channel. The applicator pad may press against the treatment area when the applicator is moved toward the application opening. At least one delivery device of the plurality of delivery devices may include an application opening that is custom fit to a size and shape of the treatment area. The at least one delivery device of the plurality of delivery devices may be configured to be custom fit. The at least one delivery device may be custom fit in at least one of an at home setting or a doctor's office. The size and shape of the treatment area may be measured with a mobile device. An ablation tool may be configured to remove at least a portion of the treatment area. The ablation tool may be positionable in each of the plurality of delivery devices.

According to a third aspect of the disclosed embodiments, a method for tissue treatments includes placing a delivery device around a treatment area. The delivery device has an application end and a delivery end. A channel extends between a delivery opening in the delivery end and an application opening in the application end. The application opening is sized to surround the treatment area so that the treatment area is accessible through the channel. The method also includes discharging a treatment through the delivery opening so that the treatment is contained in the channel. The method also includes inserting an applicator through the delivery opening. The method also includes moving the applicator toward the application opening to compress the treatment contained in the channel against the treatment area.

In some embodiments of the third aspect, an applicator pad is located in the application end adjacent the application opening to absorb the treatment delivered though the channel. The method may also include pressing the applicator pad against the treatment area with the applicator. The method may also include custom fitting the application opening to a size and shape of the treatment area. The method may also include measuring the size and shape of the treatment area with a mobile device. The method may also include removing at least a portion of the treatment area with an ablation tool. The method may also include positioning the ablation tool in the delivery device.

According to a fourth aspect of the disclosed embodiments, a delivery device for tissue treatments includes an application end and a delivery end. A channel extends between a delivery opening in the delivery end and an application opening in the application end. The application opening is custom fit to surround a treatment area so that the treatment area is accessible through the channel. The delivery end is configured to receive an infrared treatment that is delivered through the channel to the treatment area at the application opening.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a view of a kit for tissue treatments including a plurality of delivery devices, an applicator, a treatment container, and an ablation tool;

FIG. 2 is a side view of another embodiment of a delivery device having an applicator pad positioned therein and another embodiment of an applicator;

FIG. 3 is a flowchart of a method for applying a tissue treatment.

FIG. 4 is a view of a mobile device positioned to measure the treatment area of the tissue;

FIG. 5 is a schematic view of the mobile device communicating with a modeling device to build the delivery device;

FIG. 6 is a side view of the delivery device of FIG. 1 positioned adjacent to a treatment area of the tissue and the treatment container positioned to deliver treatment into the delivery device;

FIG. 7 is a side view of the applicator inserted into the delivery device to press the treatment against the treatment area; and

FIG. 8 is a side view of the ablation tool inserted into the delivery device to remove at least a portion of the treatment area.

### DETAILED DESCRIPTION

For the purposes of promoting an understanding of the principles of the disclosure, reference will now be made to a number of illustrative embodiments illustrated in the drawings and specific language will be used to describe the same.

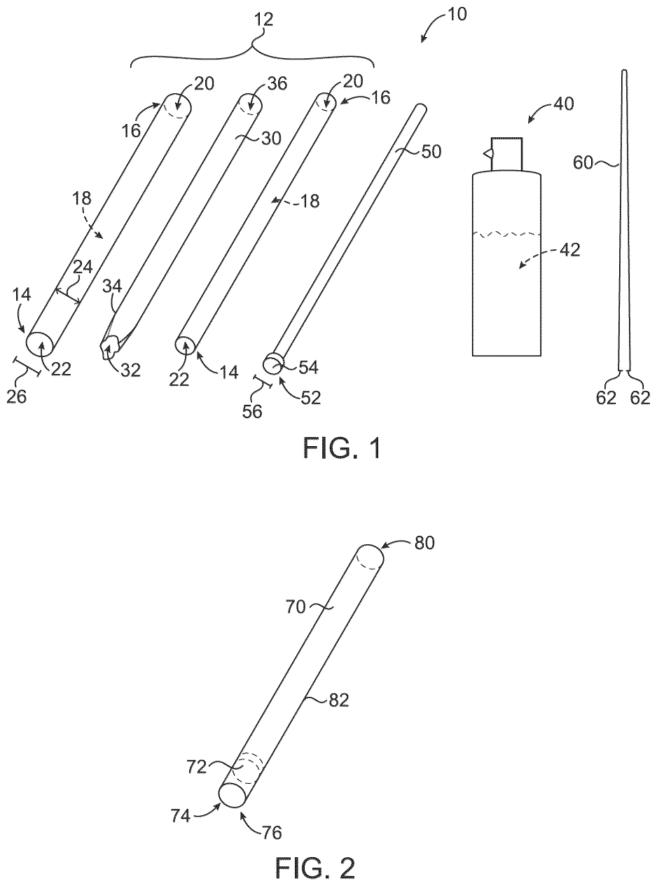

Referring to FIG. 1, a kit 10 for tissue treatments can be utilized to treat both external and internal treatment areas. In one embodiment, the kit 10 is utilized as a skin blemish remover. For example, the kit 10 is used to treat skin tags, skin blemishes, warts, skin discoloring or the like. In some embodiments, the kit 10 is used internally to treat internal tissue blemishes and/or the cauterize tissue. The kit 10 is customizable to the size and shape of the treatment area so that a treatment is applied substantially entirely to the treatment area while not being applied to healthy tissue. Additionally, the kit 10 provides a means to compress the treatment onto the treatment area.

The kit 10 includes a plurality of delivery devices 12. Each delivery device 12 includes an application end 14 and a delivery end 16. A channel 18 extends between a delivery opening 20 in the delivery end 16 and an application opening 22 in the application end 14 of each delivery device 12. The application opening 22 of each delivery device 12 is sized to surround and encircle a perimeter of a treatment area of damaged tissue so that the treatment area is accessible through the channel 18. Sizing prevents excess treatment from being applied to healthy tissue while applying treatment to substantially all of the treatment area. The delivery opening 20 of each delivery device 12 is configured to receive a treatment so that the treatment is contained in the channel 18. As used herein “contains” includes substantially retaining the treatment in the channel 18 while allowing some degree of treatment leakage from the channel 18. In some embodiments, the channel 18 of each delivery device 12 is cylindrical and has diameter 24. Each delivery device 12 has a different sized application opening 22 to accommodate different sized treatment areas. That is, a diameter 26 of the application opening 22 of each delivery device 12 differs. In some embodiments, the diameter 26 of each application opening 22 is equal to the diameter 24 of the corresponding channel 18. In some embodiments the delivery device flares outward or inward at the application opening 22 so that the diameter 26 of the application opening 22 is not equal to the diameter 24 of the corresponding channel 18. In one embodiment, at least the application opening 22 is not cylindrical and is sized to the treatment area, as shown in deliver device 30. Delivery device 30 includes a non-circular application end 32 that tapers at a tapered section 34 to a cylindrical delivery end 36. It will be appreciated that, in some embodiments, the entire deliver device 12 is non-cylindrical.

In the exemplary embodiment, the kit 10 contains a plurality of delivery devices 12, each with a different sized application opening 22. In such an embodiment, the plurality of delivery devices are sold together in the kit 10. As such, the user selects the appropriate size at the time of use. In another embodiment, the kit 10 includes only one delivery device 12 or a plurality of delivery devices 12 having the same sized application opening 22. In such an embodiment, various kits 10 of different sizes are sellable at a retailer. For example, a first kit 10 is sold with only small delivery devices 12, whereas a second kit 10 is sold with only medium or large delivery devices 12. As such, the user selects the appropriate size at the time of purchase.

In some embodiments, at least one delivery device 12 is configured to be custom fit, as described in more detail below. In one embodiment, at least one of the plurality of delivery devices 12 is customizable. In some embodiments, a kit 10 includes an individual customizable delivery device 12. In other embodiments, a kit 10 includes a plurality of customizable delivery devices 12. In yet another embodiment, the kit 10 is utilized at a physician's office where the delivery devices 12 are custom made. In a further embodiment, the kit is utilized at home where the delivery devices are custom made. In one embodiment, the delivery device is customized at the point of purchase.

A treatment container 40 is provided with a treatment 42 therein. In one embodiment, the treatment 42 is at least one of liquid nitrogen or nitrous oxide. In some embodiments, the treatment 42 is any chemical configured to treat a treatment area. In an exemplary embodiment, the treatment container 40 is a pressurized canister 44 having a nozzle or hose 46 for discharging the treatment 42 into the delivery opening 20 of the delivery device 12 when the application end 14 of the delivery device 12 is positioned adjacent to the treatment area so that the treatment area is contained within the application opening 22.

In one embodiment, the delivery opening 20 of the delivery device 12 is configured to receive a frequency treatment from a frequency device, for example a frequency device at a physician's office or a home frequency device. In some embodiments, the frequency treatment is one of an infrared, a sonic, an optical, or other frequency treatment. The frequency treatment is delivered through the channel 18 to the treatment area at the application opening 22. In such an embodiment, the kit 10 may not include the treatment container 40.

The kit 10 includes an applicator 50 having an application end 52. In the exemplary embodiment, the application end 52 includes a plunger 54. The applicator 50 is configured to be inserted through the delivery opening 20 of the delivery device 12 and moved toward the application opening 22 to apply a treatment 42 discharged into the channel 18 against the treatment area by applying pressure to the treatment 42 in the channel 18 with the plunger 54. In some embodiments, the plunger 54 is formed from a fiber swab, for example a cotton swab. In some embodiments, the plunger 54 is formed from a plastic, rubber, composite, or the like, or combinations thereof. It will be appreciated that, in some embodiments, the plunger 54 is formed from a solid material having a fiber, for example cotton, surface. In an exemplary embodiment, a diameter 56 of plunger 54 is sized to the internal diameter 24 of the channel 18 of at least one delivery device 12. In some embodiments, the plunger 54 is flexible and configured to be movable through any of the plurality of delivery devices 12. In such an embodiment, the diameter 56 of the plunger 54 is at least sized to the largest diameter delivery device 12 and capable of flexing to fit in the other sized diameter deliver devices 12.

In the embodiment of the delivery device 30 with the non-circular application end 32, the tapered section 34 tapers to the cylindrical delivery end 36 to receive the plunger 54. In such an embodiment, the channel 18 is configured to be filled with treatment 42 from the application end 32 of the delivery device 30 toward the cylindrical delivery end 36 of the delivery device 30 to a point 58 beyond the tapered section 34 of the delivery device 30. The plunger 54 of the applicator 50 is configured to be moved through channel 18 to the tapered section 34, in one embodiment.

The kit 10 includes an ablation tool 60. In some embodiments, the ablation tool 60 is optional. In some embodiments, the ablation tool 60 is provided by a healthcare provider. The exemplary embodiment includes an ablation tool 60 having a pair of blades 62 configured to pinch and cut the treatment area. In some embodiments, the ablation tool 60 includes a single blade 62. In some embodiments, the ablation tool 60 is configured to burn the treatment area. For example, in some embodiments, the ablation tool 60 includes an electrode for burn and/or cauterizing the tissue. In an exemplary embodiment, the ablation tool 60 is configured to be inserted into the delivery device 12 from the delivery end 16 to the application end 14. In some embodiments, the ablation tool 60 is applied to the treatment area without the delivery device 12.

Referring now to FIG. 2, another embodiment of a delivery device 70 includes an applicator pad 72 positioned therein. The applicator pad 72 is positioned at an application end 74 of the delivery device 70 adjacent an application opening 76. In the exemplary embodiment, a space 78 is defined between the application opening 76 and the applicator pad 72. The treatment area is configured to be contained within the space 78. In some embodiments, the applicator pad 72 absorbs the treatment 42 delivered though the channel 18. The applicator pad 72 presses against the treatment area when the applicator 50 is moved toward the application opening 76. In some embodiments, the channel 18 is filled with treatment 42 from the application end 74 of the delivery device 70 toward a delivery end 80 of the delivery device 70 to a point 82 beyond the applicator pad 72. The applicator pad 72 is compressed against treatment area with applicator 50 to treat the treatment area.

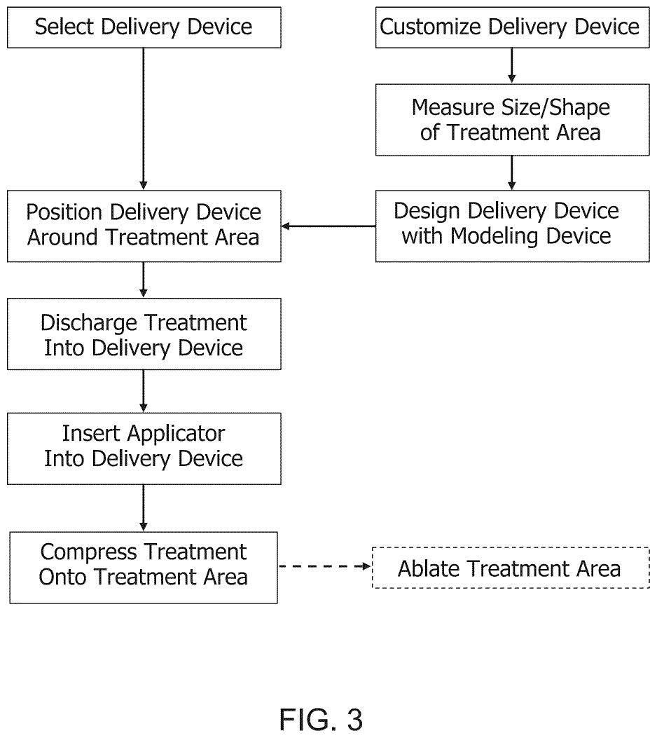

FIG. 3 is a flowchart of a method 100 for applying a tissue treatment using the kit 10. AT block 102, a delivery device 12 is selected from the kit 10. For example, in one embodiment, a delivery device 12 is selected from one of the plurality of delivery devices 12. In one embodiment, an individual delivery device 12 is purchased from a retailer or provided by a physician. At block 104, a delivery device 12 is customized for the user. For example, the application opening 22 is custom fit to a size and shape of the treatment area. It will be appreciated that any measuring device is suitable for measuring the size and shape of the treatment area.

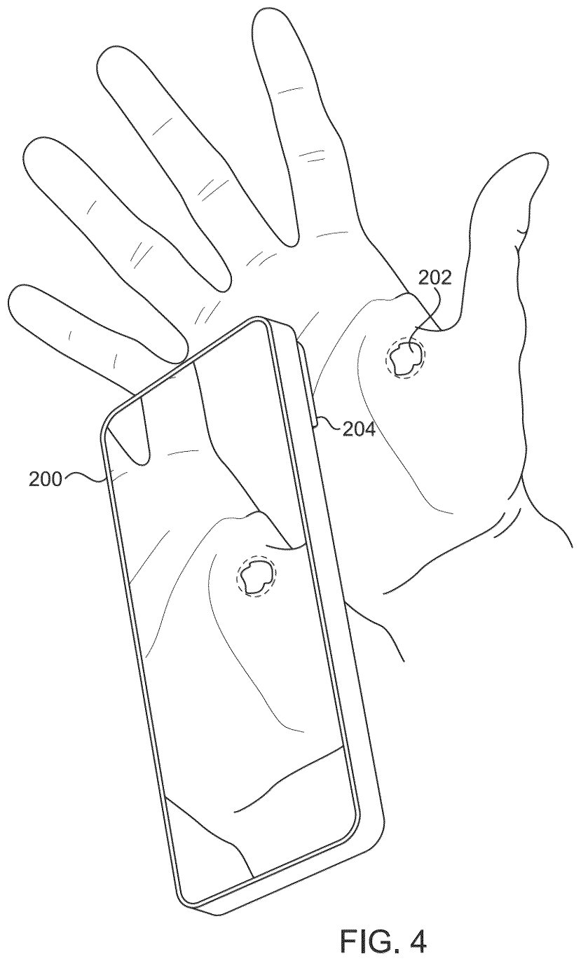

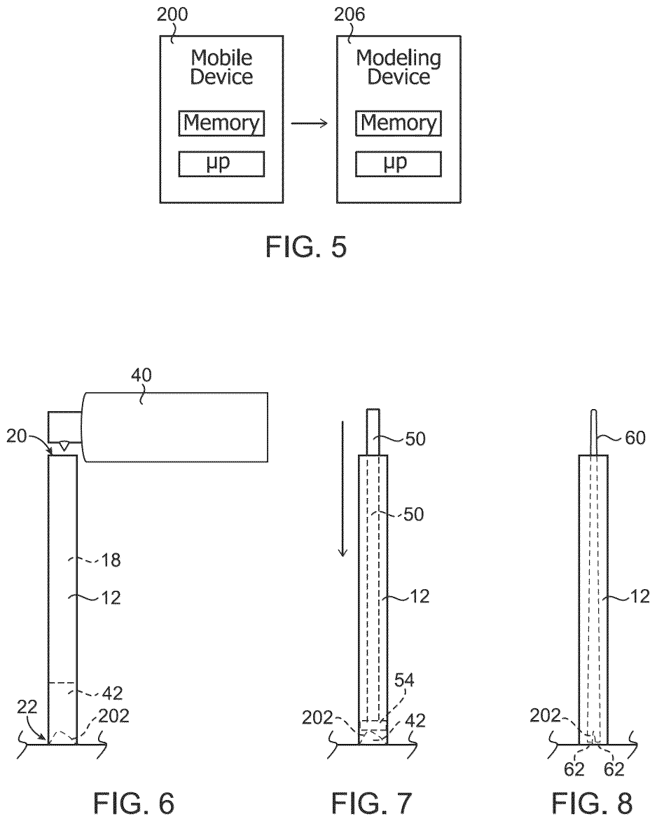

Referring to FIG. 4, in one embodiment, a mobile device 200 is positioned adjacent the treatment area 202 to measure the size and the shape of the treatment area 202, at block 106. For example, an application on the mobile device 200 utilizes a camera 204 of the mobile device 200 to measure the treatment area 202. Referring to FIG. 5, at block 108, the mobile device 200 communicates with a modeling device 206 to custom build the delivery device 12. In particular, the application opening 22 of the delivery device 12 is custom sized to the size and shape of the treatment area 202. In an embodiment, where the treatment area 202 is measured without the mobile device 200, the measurements of the treatment area 202 are otherwise input into the modeling device 206. For example, in some embodiments, the measurements of the treatment area 202 are manually input into the modeling device 206, in some embodiments. In other embodiments, the design of the delivery device 12 is manually created using an app on the mobile device 200, the modeling device 206, and/or another computer.

In some embodiments, the delivery device 12 is molded or three-dimensionally printed by the modeling device 206. In some embodiments, the modeling device 206 is provided at a physician's office, a healthcare facility, or a retailer. In some embodiments, the modeling device 206 is a home modeling device. In some embodiments, the measurements of the treatment area are sent to a remote facility having modeling devices 206 for customizing the delivery device 12. In such embodiments, the customized delivery device 12 is sent to the user's home or to a physician's office after customization for later use in treating the treatment area 202.

Referring back to FIG. 3, the selected delivery device 12 is positioned around the treatment area 202, at block 110. Referring to FIG. 6, the delivery device 12 is positioned adjacent to a treatment area 202 so that the treatment area 202 is substantially contained in the application opening 22. The treatment container 40 is positioned to deliver the treatment 42 into the delivery opening 20 of the delivery device 12. At block 112 of FIG. 3, the treatment 42 is discharged through the delivery opening 20 so that the treatment 42 is contained in the channel 18 at the application opening 22 adjacent to the treatment area 202.

An applicator 50 is through the delivery opening 20, at block 114. Referring to FIG. 7, the applicator 50 is inserted into the delivery device 12 to press the treatment 42 with the plunger 54 against the treatment area 202. Moving the applicator 50 toward the application opening 22 compresses the treatment 42 contained in the channel 18 against the treatment area 202, at block 116. In the embodiment of delivery device 70 (shown in FIG. 2), the applicator pad 72 absorbs at least a portion of the treatment 42 delivered though the channel 18. In such an embodiment, the method 100 optionally includes pressing the applicator pad 72 against the treatment area 202 with the applicator 50. In one embodiment, the method 100 of FIG. 3 optionally includes ablating the treatment area 202, at block 118. Referring to FIG. 8, the ablation tool 60 is inserted into the delivery device 12 to remove at least a portion of the treatment area 202. In some embodiments the ablation tool 60 is utilized without the delivery device 12.

Any theory, mechanism of operation, proof, or finding stated herein is meant to further enhance understanding of principles of the present disclosure and is not intended to make the present disclosure in any way dependent upon such theory, mechanism of operation, illustrative embodiment, proof, or finding. It should be understood that while the use of the word preferable, preferably or preferred in the description above indicates that the feature so described can be more desirable, it nonetheless cannot be necessary and embodiments lacking the same can be contemplated as within the scope of the disclosure, that scope being defined by the claims that follow.

When terms of degree such as “generally,” “substantially,” and “about” are used herein in connection with a numerical value or a qualitative term susceptible to a numerical measurement, it is contemplated that an amount that is plus or minus 10 percent, and possibly up to plus or minus 20 percent, of the numerical value, is covered by such language, unless specifically noted otherwise, to at least account for manufacturing tolerances. Otherwise, a suitable definition for “generally,” “substantially,” and “about” is largely, but not necessarily wholly, the term specified.

In reading the claims it is intended that when words such as “a,” “an,” “at least one,” “at least a portion” are used there is no intention to limit the claim to only one item unless specifically stated to the contrary in the claim. When the language “at least a portion” and/or “a portion” is used, the item can include a portion and/or the entire item unless specifically stated to the contrary.

It should be understood that only selected embodiments have been shown and described and that all possible alternatives, modifications, aspects, combinations, principles, variations, and equivalents that come within the spirit of the disclosure as defined herein or by any of the following claims are desired to be protected. While embodiments of the disclosure have been illustrated and described in detail in the drawings and foregoing description, the same are to be considered as illustrative and not intended to be exhaustive or to limit the disclosure to the precise forms disclosed. Additional alternatives, modifications and variations can be apparent to those skilled in the art. Also, while multiple inventive aspects and principles have been presented, they need not be utilized in combination, and many combinations of aspects and principles are possible in light of the various embodiments provided above.

1. A system for tissue treatments comprising:
a delivery device having an application end and a delivery end, wherein a channel extends between a delivery opening in the delivery end and an application opening in the application end, wherein the application opening is sized to surround a treatment area so that the treatment area is accessible through the channel, and wherein the delivery opening is configured to receive a treatment so that the treatment is contained in the channel.

2. The system of claim 1, further comprising an applicator configured to be inserted through the delivery opening and moved toward the application opening to apply the treatment contained in the channel against the treatment area.

3. The system of claim 2, wherein the applicator presses the treatment against the treatment area when the applicator is moved toward the application opening.

4. The system of claim 2, further comprising an applicator pad located in the application end adjacent the application opening, wherein the applicator pad absorbs the treatment delivered though the channel, and wherein the applicator pad presses against the treatment area when the applicator is moved toward the application opening.

5. The system of claim 1, wherein the application opening is custom fit to a size and shape of the treatment area.

6. The system of claim 1, further comprising an ablation tool configured to remove at least a portion of the treatment area.

7. A kit for tissue treatments comprising:
a plurality of delivery devices, wherein each delivery device includes an application end and a delivery end, wherein a channel extends between a delivery opening in the delivery end and an application opening in the application end, and wherein the application opening of each delivery device is sized to a treatment area so that the treatment area is accessible through the channel,
a treatment container that contains a treatment, wherein the treatment container is configured to discharge the treatment through the delivery opening so that the treatment is contained in the channel.

8. The kit of claim 7 further comprising an applicator configured to be inserted through the delivery opening and moved toward the application opening to compress the treatment contained in the channel against the treatment area.

9. The kit of claim 8, wherein the applicator presses the treatment against the treatment area when the applicator is moved toward the application opening.

10. The kit of claim 8, further comprising an applicator pad located in the application end adjacent the application opening, wherein the applicator pad absorbs the treatment delivered though the channel, and wherein the applicator pad presses against the treatment area when the applicator is moved toward the application opening.

11. The kit of claim 7, wherein each delivery device of the plurality of delivery devices has a different sized application opening to accommodate different sized treatment areas.

12. The kit of claim 7, wherein at least one delivery device of the plurality of delivery devices includes an application opening that is custom fit to a size and shape of the treatment area.

13. The kit of claim 7, further comprising an ablation tool configured to remove at least a portion of the treatment area.

14. The kit of claim 13, wherein the ablation tool is positionable in each of the plurality of delivery devices.

15. A method for tissue treatments comprising:
placing a delivery device around a treatment area, wherein the delivery device has an application end and a delivery end, wherein a channel extends between a delivery opening in the delivery end and an application opening in the application end, and wherein the application opening is sized to surround the treatment area so that the treatment area is accessible through the channel, and
discharging a treatment through the delivery opening.

16. The method of claim 15, further comprising:
inserting an applicator through the delivery opening, and
moving the applicator toward the application opening to compress the treatment contained in the channel against the treatment area.

17. The method of claim 16, wherein an applicator pad is located in the application end adjacent the application opening to absorb the treatment delivered though the channel, and wherein the method further comprises pressing the applicator pad against the treatment area with the applicator.

18. The method of claim 15, further comprising custom fitting the application opening to a size and shape of the treatment area.

19. The method of claim 15, further comprising removing at least a portion of the treatment area with an ablation tool.

20. A delivery device for tissue treatments comprising:
an application end,
a delivery end, and
a channel extending between a delivery opening in the delivery end and an application opening in the application end,
wherein the application opening is custom fit to surround a treatment area so that the treatment area is accessible through the channel, and
wherein the delivery end is configured to receive a frequency treatment that is delivered through the channel to the treatment area at the application opening.
